(19) United States
(12) Patent Application Publication
(10) Pub. No.: US 2006/0251704 A1
(43) Pub. Date: Nov. 9, 2006

(54) Methods and devices for efficacious treatment of aphthous ulcers

(76) Inventor: Edward D. Lin, Osprey, FL (US)

Correspondence Address: RAY L. WEBER;RENNER, KENNER, GRIEVE, BOBAK, TAYLOR & WEBER, 400 First National Tower, Akron, OH, 44308, US

(21) Appl. No.: 11/122,884

(22) Filed: May 4, 2005

(51) Int. Cl. A61K 9/70; A61K 8/18; A61K 36/48; A61C 5/00

(52) U.S. Cl. 424/443; 424/757; 424/49; 433/217.1

(57) ABSTRACT

Methods and devices for effectively treating aphthous ulcers are disclosed. The invention employs the removal of the plaque layer of the aphthous ulcer to accelerate healing. Before the plaque layer is removed, the ulcer may be initially anesthetized, by rubbing with an appropriate material and/or compound. Subsequently, an appropriate medicament may be applied to accelerate healing, if desired. The invention teaches the structure and use of applicator/dispenser system employing easily crushable ampoules, containing an aesthetic, and other appropriate medicaments. The invention further teaches the application of slippery surfaces to teeth, teeth appliances, and oral cavity surfaces to inhibit the generation of aphthous ulcers, and to speed their healing. The application, in the form a gel or film, may include a sustained release medicament. Finally, the invention contemplates the use of replaceable and disposable tips for power toothbrushes and the like to aid the treatment of the aphthous ulcers, and the use of dendrimer technology to provide sustained and concentrated local drug action.

### TECHNICAL FIELD

This invention relates to topical therapeutics, specifically, methods and devices for the treatment of mucocutaneous ulcerations of various causes. 0

### BACKGROUND ART

Recurrent aphthous stomatitis (RAS) or aphthous ulcer is a very common condition that typically starts in childhood or adolescence. A focal point of damaged oral mucosa quickly enlarges to readily visible small, round, or ovoid ulcers with circumscribed margins, erythematous halos, and yellowish to grey floors. Invariably painful to touch, and aggravated by food in the mouth, the cause of RAS is not entirely clear, and may be the manifestation of a group of disorders of quite different etiology, rather than a single entity.

Despite many studies trying to identify a causal microorganism, RAS does not appear to be infectious, contagious, or sexually transmitted. The coloration of the ulcer crater is due to a plaque of necrotic tissue, fibrinous exudates, inflammatory cells and cytotoxic mediators including proteases, tumor necrosis factor, and a host of opportunistic pathogens multiplying within. A variety of bacteria, spirochetes, viruses and yeasts have been found in the fibrinous crater of the ulcers but not with consistency or correlated with serum antibody levels to attribute a causal relationship. Immune mechanisms appear at play in persons with a genetic predisposition to oral ulceration; about one third of patients with RAS have a positive family history. Phagocytic and cytotoxic T cells likely aid in destruction of oral epithelium that is directed and sustained by local cytokine release.

The three main clinical types of RAS are (1) minor aphthous ulcers, which comprise 80% of all RAS and last 7 to 14 days), (2) major aphthous ulcers which are typically 1 cm in size or larger, heal in 1040 days and can affect even keratinized surfaces of the oral cavity such as the tongue, and (3) herpetiform ulcers, which are characterized by multiple pin▴head sized lesions that recur so often as to be virtually continuous, and which are the most painful of all and most commonly found in older, predominantly female patients.

Predisposing factors may include the following:

Deficiencies of iron, folic acid (folate), or vitamin B

Physiologic or emotional stress

Trauma—Biting of the mucosa and wearing of dental appliances such as orthodontic braces

Endocrine factors in some women—clearly related to the progesterone level fall during menstrual cycle, and which may temporarily regress in pregnancy

Allergies to food—Occasionally underlie RAS; incidence of atopy is high

Sodium lauryl sulfate (SLS)—A detergent in some oral healthcare products; may produce oral ulceration

Immune deficiencies—Ulcers similar to RAS may be seen in HIV and some other immune defects

Drugs, especially NSAIDs, alendronate, and nicorandil—May produce lesions clinically similar to RAS.

An estimated 60 million Americans suffer from RAS, spending $146 million in treatment each year. The incidence in the general population is 20%, but as high as 50% in college students. There are a number of over-the-counter medications for RAS including Kank-A, Blistex, Zilactin and Camphophenique, all of which provide very limited symptomatic relief via a local anesthetic such as procaine or lidocaine, counterirritants such as menthol or camphor, or miscellaneous emollients. Prescription treatments include topical steroids (most commonly, triamcinolone acetonide in carboxymethyl cellulose paste under the brand name of Kenalog and Orabase) administered 4 times daily, various antibiotic preparations in the form of rinses, troches or patches, local injection of steroids and the use of systemic immunomodulators such as thalidomide. Jack and White in US Patent application US2003/0187048 A1 disclosed highly diluted histamine phosphate as an effective topical treatment for a variety of mucous membrane and skin conditions ranging from aphthous ulcers to conjunctivitis, skin burns to decubiti. Most recently, amlexanox (Aphthasol® from Access Pharmaceutical), an inhibitor of inflammatory mediators, has been approved for prescription in the US and Europe.

In the case of antibiotics, since no specific organism has been shown to be clearly a causative agent, such treatment carries the risk of further disrupting the normal balance of the oral bioflora and inducement of microbial resistance. In the case of steroid therapy, the drug nonspecifically reduce the degree of inflammation and blunt the body's own destructive immune processes that are believed to play a role in the pathogenesis. However, steroids also depress the body's ability to fight off secondary infections that also play a role in the disease process, as well as being generally immunosuppressive, rendering the patient vulnerable to other complications. Thalidomide carries the risks of teratogenicity and neuropathy, accounting for the reluctance of many physicians to use it. Amlexanox appears to offer an improved, more specific approach; even so, only 65.6% of patients have healed ulcers by the 6th day, compared to 54.7% in the control receiving the vehicle. Of patients treated with amlexanox, 82.8% are pain free by the 6th day compared to 72.8% in the control group. All of the above require multiple applications of treatment each day. All in all, the current state of the art in treatment for RAS underscores the fact that all treatment solutions have overlooked an important cornerstone to overall treatment. As a direct consequence, even though some of the approaches are beneficial in theory, their efficacy remains severely compromised or undermined.

The inventor herein has observed through many and repeated clinical observations in patients that once a mucosal breakdown occurs, a blanket of microbes quickly aggregate and multiply giving rise to the clinically evident and growing ulcer. If at this earliest phase of aphthous ulcer formation, the ulcer is completely cleaned and cleared of this microbial plaque, then the ulcer quickly vanishes and the pathogenesis is terminated within one to at most a few days, depending on the stage of intervention. Since the presence of the plaque plays a major role in the early development and persistence of the ulcer, the sooner the intervention in its removal, the faster the healing. This is consistent with the general belief that exaggerated cytotoxic activity triggered by the initial trauma or presence of microbial proteins leads to a vicious cycle of tissue destruction before the process slowly terminates after its extended and painful natural course. In spite of the extensive teachings of the prior art to deal with RAS, there is no suggestion of the crucial importance of removing the plaque covering the ulcer as a fundamental first-step in therapy as presented herein.

After a careful search, the inventor is unable to find any treatment on the market that is directed at removing the fibrinous plaque that harbors the organisms that play either a causative role or are retarding or aggravating the healing process by their presence. All rinses, patches, gels, troches and other therapeutic modalities are directed at treatment over and through the plaque itself. Since the plaque serves both as a sanctuary to opportunistic and harmful organisms and at the same time serves as a physical barrier to therapy, it is the singular reason even the best treatment modalities of the prior art are not very effective. In contrast, this invention provides a range of low-cost and exceptionally effective systems for plaque removal, thereby eliminating instantly the offending source and barrier to healing. The result is a quick arrest and reversal of the natural progression of RAS during the development phase, and immediate acceleration towards a rapidly healed state.

### SUMMARY OF THE INVENTION

Given the aforedescribed current state of the art, it is an aspect of this invention to provide an effective and yet economical treatment for aphthous ulcer that is not dependent on a prescription, and is directed at removing the offending and aggravating plaque from the crater.

It is another aspect of this invention to provide a variety of effective treatment systems that are easy for the patient to use and apply in accomplishing the above goal.

It is yet another aspect of this invention to provide a variety of effective treatment systems that are easy for the patient to use and apply in the delivery of both prescription and over-the-counter medication to the oral cavity.

To simplify the application of medicaments through crushable ampoules, it is still another aspect of this invention to provide a dispensing structure that would greatly reduce the finger force needed to break the ampoules.

Yet a further aspect of this invention is to provide for a novel applicator that can quickly coat orthodontic appliances with a layer of protective mucoadherent gel such as hydroxypropyl cellulose (KlucelO).

Yet another aspect of this invention is to extend the mucoadherent gel technology to provide a more effective protective coating for the esophagus against gastric acid erosion in gastroesophageal reflux disease (GERD).

Further, because of the painful nature of aphthous ulcers, most patients are reluctant to touch the lesion, and since they are unaware of the curative effect cleaning would have upon the natural progression of the disease, there is no motivation as yet to actively do what it takes to clean off the necrotic plaque that covers the ulcer. Therefore, it is yet another important aspect of this invention to apply the gate theory of pain, in combination with a motorized device to relieve the discomfort associated with cleaning off the plaque. In this regard, it is well known that small-diameter nerve fibers carry pain stimuli through a aegate mechanism's but larger-diameter nerve fibers going through the same gate can inhibit the transmission of the smaller nerves carrying the pain signal. Therefore, this invention provides a vibratory or rotary probe, the distal end of which carries a disposable and medicated atraumatic cleaning surface. When applied to the ulcer crater as part of the cleaning process, not only do the proprioceptive signals overwhelm the sensory pathways and keep the pain gates (that would otherwise have transmitted pain signals) closed, the cleaning can be accomplished in an instant and more effectively.

The foregoing and other aspects of the invention that will be further discussed in the detailed description of the invention provided hereinafter are achieved by a method for treating aphthous ulcers and the like, comprising the step of removing the plaque layer of dead tissue, fibrinous exudate and microbes.

Other aspects of the invention that will become apparent herein are achieved by the method for improving intraoral milieu comprising the steps of applying a hydrophilic slippery coating to at least one of the surfaces of teeth, the surface of dental appliances, or other intraoral surface.

Still further aspects of the invention which will become apparent herein, are achieved by a cleaning and therapeutic device having crushable ampoules for the treatment of aphthous ulcers, comprising: a tubular container; a crushable ampoule maintained within said tubular container; and wherein said tubular container has a first inner surface area configured to deliver focused pressure to surface of said ampoule to effect crushing thereof.

Additional aspects of the invention, which will be treated herein, are attained by a device for treating aphthous ulcers, comprising: a motorized handle, similar to that of a power toothbrush, having a receiving end for removable and replaceable working tips comprising a soft brush, a soft cover, and a soft cleaning tip, wherein at least certain of said tips release medicaments.

A further aspect of the invention is to deploy dendrimer technology to nanoengineer molecules having antimicrobial or other desirable properties to achieve high local drug concentration and sustained action.

### BRIEF DESCRIPTION OF DRAWINGS

For a complete understanding of the objects, techniques and structure of the invention, reference should be made to the following detailed description and accompanying drawings wherein:

FIG. 1 is a perspective view showing one embodiment of the instant invention in the form of dual-ended premedicated cotton-tipped swabs, one end being premedicated with a local anesthetic while the other is impregnated with a tissue-compatible fine abrasive such as sodium bicarbonate or dentifrice;

FIG. 2 shows yet another embodiment of the invention in the form of a dual-tipped applicator in a hollow stem containing crushable ampoules that deliver medicaments in sequence;

FIG. 3 shows a cross section of an applicator stem of the prior art and as shown in FIG. 2, taken along line 3-3;

FIG. 4 shows a cross section of the applicator stem in FIG. 2 taken along the line 3-3, when modified to an easily-crushable new dispensing system as disclosed in detail herein;

FIG. 5 shows a cross section view of one side of the oral cavity with three ulcers at common locations, and wherein two therapeutic mucoadhesive patches are shown that deliver desired medicaments;

FIG. 5A is a cross sectional view of a multilayered patch that may cover, shield and medicate an aphthous ulcer;

FIG. 6 shows a perspective view of a dispenser for delivering a viscous coating to orthodontic appliances to reduce their trauma potential and ability to harbor pathogenic microbes; and

FIG. 7 shows an electrically driven mechanized handle delivering rotary or vibratory movements to its tip, which mates with a disposable cleaning surface.

### DETAILED DESCRIPTION OF THE PREFERRED EMBODIMENT FOR CARRYING OUT THE INVENTION

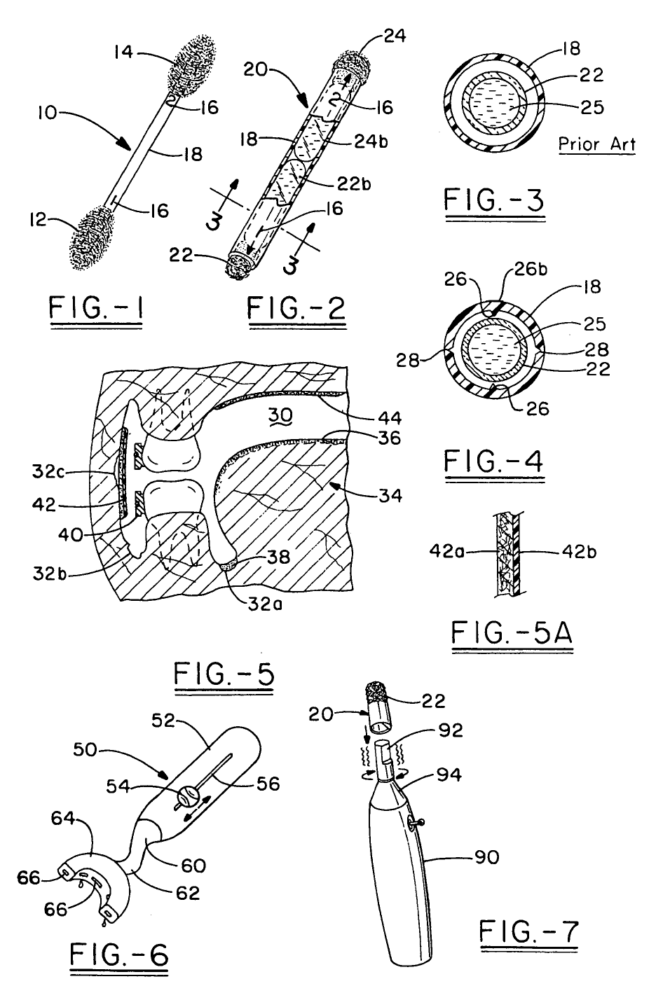

FIG. 1 shows a perspective view of one embodiment of the instant invention in the form of a premedicated dual cotton-tipped swab 10. One end 12 of the swab is premedicated with a local anesthetic such as procaine or lidocaine while the other end 14 is impregnated with a tissue compatible fine abrasive or a mixture of such. These would include, though not be limited to, sodium bicarbonate, dicalcium phosphate, insoluble sodium metaphosphate, calcium pyrophosphate, calcium orthophosphate, calcium carbonate, magnesium carbonate, or one of a variety of silicates and dehydrated silica gels, and ultra fine aluminum hydroxide. All subsequent reference to “abrasive” or “abrasives” shall be understood to mean suitably fine histocompatible particulate matter that helps to remove a thin layer of cellular debris and would not injure tissue when used in the manner described. Indicia markings 16 such as a numerical sequence (Step 1 then Step 2) on either the shaft 18 of the cotton-tip applicator or even on the cotton tips themselves can help guide the user in the optimum sequence of use of the premedicated tips. Alternatively, a dilute food dye (such as light green or pink) can be added to one or both tips to help distinguish by color code the chemical ingredient in one end from the other. In actual use, the patient would moisten the local anesthetic end and apply it to the ulcer crater and its rim. Alternatively, the tip can be pre-moistened and ready to use and sealed in a variety of suitable coverings such as a polymer film or foil pouch; these packaging methods would be readily apparent to those skilled in the art. Once the numbing effect commences in 15 to 30 seconds, the patient can then gently rotate the shaft of the cotton swab to begin the process of cleaning off the plaque.

The cleaning process enters its final phase of an additional 30 to 45 seconds when the patient reverses the swab ends to apply the fine abrasive end to the ulcer crater and complete the rotary or frictional cleaning process until a clean pink ulcer bed emerges and all remnants of the plaque are gone. The used cotton applicator is then discarded. This process can be repeated one or more times a day to maintain a pink and clean ulcer bed. Rapid healing and disappearance of all symptoms including pain is typically achieved within one to three days instead of the usual 7 to 14 days. The earlier the intervention, the more dramatic and speedy the healing. If the ulcer is cleaned when the crater first begins and has grown to no larger than 3 mm in diameter, the ulcer pain stops almost immediately and the crater vanishes in less than a day. A repeat treatment is often unnecessary.

Since cleaning the ulcer of its plaque is the central goal of therapy, any mechanical device capable of gently removing the plaque can serve the purpose.

Therefore, such a device can take multiple forms and be constructed of a variety of materials as would be evident to those skilled in the art. The cleaning tip can be made of cotton or fine polymer fibers, gauze or even soft sponge or foam-like material. The local anesthetic is to alleviate the discomfort associated with the initial cleaning and the fine abrasive(s) is to facilitate the gentle removal of the plaque material. They are desirable, though not essential components of the treatment process. Therefore, a single-tipped or dual-tipped cotton applicator free of medicament can be used to substantially accomplish the plaque removal, albeit attended with a higher degree of temporary discomfort.

FIG. 2 shows yet another embodiment of the present invention in the form of a tubular swab 20 including one or more crushable ampoules of medicaments for sequential therapy. Tip 22 is comprised of a plug of soft gauze-like material and designed to receive medicament from ampoule 22b, such as a local anesthetic, a counter irritant such as menthol or an herbal extract such as glycyrrzhiza from licorice with an analgesic property of unknown mechanism. Tip 24 is similarly constructed but designed to deliver the medicaments in ampoule 24b, which can be a hydrogen peroxide or other antimicrobial solution or gel in instant or sustained release form. Similarly, other suitable medicaments including proteolytic enzymes such as papain, urokinase, etc., may also be used to aid the removal of large and thick more-difficult-to-remove plaques.

Instructional indicia 16 can optionally be imprinted on the shaft 18.

Although FIG. 2 illustrates two distinct medicament ampoules, each with its respective tip, it is also feasible to have two distinct ampoules with just one tip or two identical ampoules with one tip or two tips; they will all substantially accomplish the goal of removing the aphthous plaque.

It should be readily apparent to those skilled in the art that all of the aforementioned classes of medicaments can be applied independently to an unmedicated cleaning tip or be made an integral part of the cleaning tip as part of the manufacturing process. For example, instead of the local anesthetic or peroxide gel being pre-impregnated in the cleaning tip, one can dip an unmedicated cleaning tip into a vial of local anesthetic or peroxide gel to achieve the same medicated tip to be used for cleaning. Similarly, the medicaments in whatever sequence of application can be stored in discreet ampoules to be crushed open for delivery as needed for use, as shown in FIG. 2. In addition, the sequence of serial application of medicaments can be altered or substituted as desired without departing from the scope and spirit of this invention. For example, any of the following sample sequence of therapies, in addition to others not specified, but apparent to those skilled in the art, could be beneficial for aphthous ulcer therapy:

1) Clean ulcer with local anesthetic, followed by fine abrasive

2) Clean ulcer with abrasive, followed by antiseptic

3) Clean ulcer with antiseptic, followed by steroid or immune modulator

4) Clean ulcer with saline, followed by steroid

5) Clean ulcer with abrasive, followed by licorice or other herbal extract gel

6) Clean ulcer with abrasives, followed by antibiotic gel or patch

7) Clean ulcer with local anesthetic, followed by proteolytic enzyme

8) Clean ulcer with proteolytic enzyme, followed by antiseptic, etc.

FIG. 3 shows a cross section of applicator stem in FIG. 2 along line 3-3, which represents the current state of the art. Although crushable ampoules 22 in a hollow dispenser stem 18 have been around for many years, one disadvantage of the current system is that considerable finger pressure is needed to deform the hollow stem and crush the ampoule. Both the stem and the ampoule have certain minimum thickness requirements in order to maintain the structural integrity of the applicator and in many instances, to protect and isolate the liquid chemical or medicament 25 within. For example, when the medicament is ammonia, only certain glass of a minimal thickness will withstand the reactive nature of the ingredient within. The trepidation that many users have in trying to crush a glass ampoule is that broken shards within may penetrate the stem and cut their finger, and yet at the same time they realize that unless a substantial force is applied, the ampoule cannot be successfully broken to release the medicament. Furthermore, in an aging population with increasing incidence of arthritis, this task can be so difficult as to be impossible in arthritic or weak hands.

FIG. 4 shows a cross section of applicator stem in FIG. 2 along line 3-3 when modified to an easily-crushable new dispensing system. The stem 18 in this instance has a pointed structure 26 that can be in the form of a sharp pointed ridge that is continuous with the longitudinal axis of the applicator stem. When the stem is squeezed at any point along 26, the sharp point of the ridge against the ampoule substantially reduces the pressure needed to crush the ampoule. In the preferred embodiment, an additional ridge can be provided at the opposite side so that the crushing force needed to break the ampoule is further reduced. An optional indicator 26b, by color or other indicia, can optionally be provided near the gravitationally dependent end of the ampoule to aid the user to apply the pressure at the optimum position to allow full discharge of the medicament from within the ampoule. The presence of the ridges provides structural strength to the applicator stem and will enable the overall stem wall thickness to be reduced as a material-saving benefit during manufacture.

As an alternative or complement to further reducing the force needed to deform the applicator stem sufficiently to crush the ampoule within, the applicator stem can be manufactured with a structural weakness in the form of one or more linear v-shaped grooves 28 that run longitudinally. When pressure is applied, the stem will “collapse” along the grooves, making it easier to crush the ampoules between the fingers. This concept can further be extended to large-sized ampoules that contain sterile medicaments (such as povidone iodine) for prepping a large skin surface. At one end of the hollow prep stem is an absorbent sponge pad, while a large ampoule of the solution is contained within the stem. Due to the larger size, often 15 mm or greater in diameter, the force needed to crush the thicker-walled ampoule is typically greater than can be readily mustered by most hands. In this instance, dual sharp ridges 26 will make the ampoule crushing relatively effortless for all. It should be evident that sharp-pointed ridges 26 can work independently of grooves 28 and the two structures have synergistic action with each other. Such modified stem can be extruded easily via a suitably shaped die, as would be readily apparent to those skilled in the art.

FIG. 5 illustrates a cross sectional view of the oral cavity 30 showing a sublingual ulcer 32a that is covered by a thick aphthous plaque 38, a gingival ulcer 32b showing a newly cleaned ulcer crater with the plaque removed, and a buccal aphthous ulcer 32c on the cheek that is cleaned and protected by a patch 42. Buccal aphthous ulcer is frequently caused by self-inflicted accidental biting (pinch bite) or trauma from dental appliances such as orthodontic braces and brackets 40. In such instance, continued exposure to the inciting factors will delay the healing. Currently, dental professionals provide patients with strips of easily moldable dental wax that are pressed over the offending dental appliance to minimize the degree of continued irritation. Such wax rarely stays on for any significant period of time, and repeated applications throughout the day for many days is typically necessary until the healing takes place.

In contrast, using yet another embodiment of the present invention, the ulcer crater is first cleaned, and then a mucoadhesive polymer backed patch 42 is applied over the ulcer. This patch can comprise a single layer of suitable material such as hydroxypropyl cellulose or carboxymethyl cellulose matrix impregnated with beneficial medicaments including local anesthetic, antiseptic, antimicrobial and/or steroids. More preferably, as shown in FIG. 5A, it should comprise of a bilayer or multilayer sustained release patch which has a smooth polymer backing 42b and a sustained release medicament layer 42a. Such technology is well known in the sustained release transdermal, transmucosal delivery industry and will not be expounded here. The polymer film backing over the buccal ulcer not only serves as a substrate for the medicament layer but also serves a slippery “shield” against further trauma by the biting teeth or dental appliances.

In patients prone to such injuries, the dentist or orthodontist can even prophylactically provide such patches which may have little more than sustained release breath fresheners as the active ingredient. One novel advantage to the application of buccal patches 42 or palatal patches 44 is that they distance the taste substantially from the top of the tongue 34 where the taste buds 36 are primarily located. It is well known that the commonly available breath freshener strips (such as the Listerine oral strips) which are applied over the tongue are too strong for many people. The user is faced with the dilemma of choosing between an appreciable level of breath freshener, and being overpowered by the excessive taste bud stimulation. Pfizer has recently introduced a less powerful version of the Listerine strips; however, just as in therapeutic blood level of a drug, when the starting dose is low, the freshener soon becomes too low to be effective. It is not suitable socially and at formal meetings to repetitively place a fresh oral strip on the tongue because the very action requires sticking one's tongue out and also draws unwanted attention. It would be preferable to have a potent long lasting strip that does not overstimulate the taste buds.

The instant invention further offers two novel solutions that are hereby disclosed. First, the new application of palatal and buccal patches or strips on these unconventional sites make the taste of medicaments less intense and last longer because they are less in the direct path of salivary flow or are only in intermittent contact with the tongue, especially the dorsal surface of the tongue where the taste buds reside. In the case of the palatal patches, the tongue and the gravitationally draining saliva touches the palate only during certain parts of speech or swallowing, hence reducing the amount of eluent flow of the medicament and allowing a more prolonged and sustained action within the oral cavity. Since the palatal patch is not applied to taste buds directly and is not in contact with taste buds for the sustained period of time that lingual patches are, the potency of the patch can be higher. A modified applicator (resembling a typographic-correction tape reel) can be used to apply the palatal patch or the patch/strip for orthodontic braces. Applicator is also useful on nursing home patients and has the added benefit of reducing contagion and bodily fluid transmission between patient and healthcare workers. The dispenser can be via a disposable applicator head that is sponge-lined and ejected or discarded after use.

Second, as yet another embodiment of sustained release oral strips, these can be made of two or more layers. The layer in contact with the taste buds of the tongue can have a variably lower concentration, or even be devoid, of the active ingredients while the adjoining layer has the active ingredient. This would then allow a higher-than-otherwise-palatable concentration of the active ingredient to be incorporated into the oral strip. The layer in contact with the tongue should preferably have slower dissolve/disintegration characteristics so as to remain longer as a buffer layer, protecting the taste buds from undesirable stimulation. The same principle can also be applied to strips that are applied to palatal, buccal or any other surface in the oral cavity. Such a specially formulated bilayer oral strip can be color coded to help distinguish which side should be applied in contact with the mouth. It is additionally anticipated that more than one layer of flavors may be included to be released in sequence.

In the case of protective oral patches, it is preferable to have a barrier that is also highly lubricated when in the moist environment of the mouth so that the chance of additional self-inflicted trauma (as from a pinch bite) is sharply reduced. Patches in the latter category may need a more rigid backing and therefore may be marketed in a form distinct from the type that is to be used in the sulcus of the mouth where there is no risk of trauma from biting teeth and a more pliable patch would provide added comfort. In addition, antimicrobials can further be bound to the patch via ionic or molecular bonding using dendrimer technology so that a relatively small amount of antibiotic need be used, while maintaining a locally high concentration for a sustained period of time due to the bonding mechanisms in place that prevent the antibiotic from being eluded from the region of the ulcer. An additional benefit of this is that since the antibiotic is not diluted and washed away by secretions where it can present a non-lethal threat to other microbes, the likelihood of antibiotic resistance is also reduced.

FIG. 6 shows yet another embodiment of this invention directed to treat orthodontic-appliance-induced aphthous ulcers and suppressing the ability of the appliances to harbor detrimental microbes.

It is known that hydroxypropyl cellulose (KlucelO) is more mucoadherent than carboxymethyl cellulose. The solubility of hydroxypropyl cellulose is altered by esterification of the polymer by tannic and salicylic acids. Boric acid effectively crosslinks the polymers to form a film lasting up to 6 hours. This gel film is not altered by exposure to extremes in temperatures encountered in drinking hot and cold liquids. Considering the fact that tens of millions of patients with gastroesophageal reflux disease (GERD) suffer from chronic irritation of the lower esophagus by acid reflux from the gastric pouch, and that such condition can lead to esophageal erosion, ulceration and possibly cancer, it is advantageous to apply the sustained-coating formulation aforedescribed as the delivery vehicle for antacids. This novel combination formulation not only protects the esophageal lining, but also in a sustained manner releases acid-neutralizing alkaline medicaments to relieve the discomfort of acid irritation. Additionally, in patients with advanced oroesophageal inflammation, steroids, immune modulators and other medicaments may also be used in the sustained coating formulation.

A gel delivery device 50 has a handle 52 with a screw-off top 60. Top portion 60 in turn has a flexible and ergonomic neck 62 curved in such a way to ease its insertion and movement within the buccal cavity. The neck has a hollow conduit within connected to a U-shaped dispenser 64 that is sized to straddle the “track” of standard orthodontic brackets and wires. The dispenser has perforations or apertures 66 that are connected via a conduit to a gel reservoir in the handle 52. Within the handle is stored a supply of gel medicated with an antiseptic such as sustained release peroxide in a compressible pouch. The gel has adhesive properties that will enable it to stick to dental appliances for a sustained period of time. In this illustration, a tubular pouch of medicament is placed in the handle and a connection made with the dispenser conduit in the top 60 via a tubular piercing device. Such mechanisms are well known to those skilled in the art and the details are not shown. A dispensing means such as a slidable plunger 54 travels from the base of the handle towards the dispenser 64 and applies a coating of clear gel that coats the orthodontic appliances with a slippery layer, prevents food and other carious particles from sticking in between the braces and also reduces the risk for dental decay by providing sustained release of hydrogen peroxide. This and other suitable antimicrobials and antiseptics help to suppress and eradicate microbes that can incite aphthous ulcer, dental plaque formation or other undesirable consequences.

The method described herein is for illustrative purpose only. Other suitable methods of continuous or intermittent gel or paste dispensing are well known in the art and will not be further discussed or illustrated here.

Alternatively, a U-shaped dental patch can be dispensed via extrusion employing a U-shaped die, to cover the dental appliances to accomplish a similar goal.

Lastly, FIG. 7 shows yet another embodiment of the present invention in which a motorized handle, similar to those found in modern electric and ultrasonic toothbrushes, is equipped with a specialized tip, adapted to receive a mating cleaning cartridge 20, such as the type discussed with regard to FIG. 2. An electrically driven mechanized handle 90 delivers rotary or vibratory movements to its tip 92. The tip is detachable as part of the head portion 94 which is interchangeable with regular electric toothbrush heads. Attachable to the tip 92 is a disposable medicated cartridge 20 with a tip 22 for cleaning an ulcer crater. Within cartridge 20 can be placed various medicaments that aid in the cleaning of the ulcer.

Because aphthous ulcers are inherently very painful, most patients are afraid to touch the lesion, and would be reluctant to brush, rub or clean off the necrotic plaque that covers the ulcer, especially since such benefits, until this disclosure, were not known or recognized. Applying the gate theory of pain, the proprioceptive sensation delivered by a vibrating tip would “mask” or overwhelm the gate of sensory pathway, allowing quick and more thorough cleaning of the ulcer crater with little or no discomfort. It is not necessary to create a whole new appliance just for this purpose. Much like toothbrush heads are interchangeable on a power or electric toothbrush handle, a probe adaptor head can be made to fit and work with each of the many existing electric and ultrasonic toothbrushes. The probe adaptor can either be equipped with an exceptionally soft brush for cleaning off the aphthous plaque or accept disposable cleaning cartridges tips such as those illustrated in FIGS. 1 and 2. Lastly, for cleaning of large contiguous ulcers or when the ulcerations are too numerous to clean individually, a cleaning sleeve or cover, made of suitably soft material, can be pulled over an electric toothbrush head and used as a cleaning device. Such toothbrush covers or sleeves can be premedicated, or used in conjunction with suitable medicaments.

Dendrimer technology is a relatively new nanotechnology of molecular engineering, useful for targeting and delivery mechanisms. Moieties of desired characteristics can be sequentially attached to a molecular core, resulting in a synthetic molecule of certain desired electrical charge and/or structural characteristics. Since antibiotics and other medicaments exert their effects by certain molecular structural characteristics, it is possible to combine or bond at the molecular level the active moieties that provide desirable pharmacologic effects. Therefore, for antimicrobials, for example, such moieties can be structures that bind to receptors on microbes that lead to cellular lysis or inhibition of vital cellular metabolic processes. Similarly, cellular modulators that reduce pain and inflammation, chemotherapeutic agents that target transformed cells, genetic material that have a salubrious effect on physiologic or metabolic processes, etc. can be attached to dendrimers that are anchored to a stable delivery matrix or are themselves resistant to translocation, metabolic breakdown or renal excretion. Such specially engineered dendrimers and drug matrices can exert three exceptional benefits: 1) high drug concentration, 2) localized area of action, and 3) prolonged and sustained action. Where the cost of drug is high or systemic side effect and toxicity significant, this system will further enhance the cost-effectiveness and safety of such dendrimer-based therapeutic method.

Thus it can be seen that the aspects of the invention have been satisfied by a novel and fundamental approach to treating aphthous ulcers, aimed at the physical removal of the plaque material covering the ulcer, and optionally followed by treatment with various medicaments. A variety of methods and devices that aid in the accomplishment of the above goals have also been disclosed. The disclosed invention provides a new fundamental cornerstone to treatment and direct path to rapid healing of aphthous ulcers that has thus far been unobvious to those skilled in the art including physicians, dentists and manufacturers of various therapeutic modalities for this common and painful condition. That this realization has eluded others in this field may lie in the belief or presumption that the aphthous plaque is somehow similar to a scab, to be left alone as a protective covering as the wound heals. It is counterintuitive, as one attempts to relieve the patient of his suffering, to suggest a procedure or approach that can, if only briefly, increase the pain or discomfort. Further, this invention discloses a variety of novel therapeutic modalities that are not only cost-effective and dramatically efficacious but also provide pain relief in the treatment process.

It is appreciated that certain features of the invention, which are, for the sake of clarity, described in the context of separate embodiments, may also be provided in combination in a single embodiment. Conversely, various features of the invention, which are, for brevity, described in the context of a single embodiment, may also be provided separately or in any suitable partial or total combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned or listed in the prior art in this specification are herein incorporated in their entirety by reference into the specification, to the same extent as if each individual publication, patent or patent application was specifically and individually indicated to be incorporated herein by reference.

In addition, citation or identification of any reference in this application shall not be construed as an admission that such reference is available as prior art to the present invention.

The embodiments provided herein are for illustrative purposes only, and not intended to limit the spirit and scope of the invention. Other suitable forms, processes and materials can be substituted as would be apparent to those skilled in the art. Accordingly, for an appreciation of the scope and breadth of the invention, reference should be made to the following claims.

What is claimed is:

1. A method for treating aphthous ulcers and the like, comprising the step of:
removing the plaque layer of dead tissue, fibrinous exudate and microbes.

2. The method for treating aphthous ulcers and the like as recited in claim 1, wherein said step of removing comprises rubbing a fine abrasive upon the plaque layer.

3. The method for treating aphthous ulcers and the like as recited in claim 2, wherein said fine abrasive is taken from the group comprising sodium bicarbonate and dentifrice.

4. The method for treating aphthous ulcers and the like as recited in claim 1, wherein said step of removing comprises rubbing a chemical composition upon said plaque layer.

5. The method for treating aphthous ulcers and the like as recited in claim 4, wherein said chemical composition is taken from the group comprising hydrogen peroxide, povidone iodine, chlorhexidine, an antimicrobial, an anesthetic, a pain reliever, an analgesic, a counter irritant, a proteolytic enzyme, and an immune modulator.

6. The method for treating aphthous ulcers and the like as recited in claim 1, wherein said step of removing comprises rubbing a surface of soft material upon the plaque layer until the plaque layer is removed.

7. The method for treating aphthous ulcers and the like as recited in claim 6, wherein said surface of soft material comprises an atraumatic material.

8. The method for treating aphthous ulcers and the like as recited in claim 6, wherein said surface of soft material carries a medicament.

9. The method for treating aphthous ulcers and the like as recited in claim 8, wherein said medicament is taken from the group comprising an anesthetic, a dentifrice, an antimicrobial, amlexanox, a steroid, licorice root extract and herbal extract.

10. The method for treating aphthous ulcers and the like as recited in claim 9, wherein said step of removing the plaque layer comprises a first step of applying an anesthetic medicament to the plaque layer, followed by a second step of applying a second of said medicaments to said plaque layer with a rubbing action.

11. The method for treating aphthous ulcers and the like as recited in claim 8, wherein said surface of soft material is carried by a motorized device that effects the rubbing of the surface of soft material upon the plaque layer.

12. The method for treating aphthous ulcers and the like as recited in claim 11, wherein said surface of soft material comprises a removable and replaceable tip on said motorized device.

13. The method for improving intraoral milieu comprising the steps of applying a hydrophilic slippery coating to the surface of at least one of the teeth, dental appliances, the intraoral cavity.

14. The method for improving intraoral milieu as recited in claim 13, wherein said hydrophilic slippery coating carries formulations taken from the group comprising antimicrobials, mouth freshener and dentifrice.

15. The method for improving intraoral milieu as recited in claim 14, wherein said hydrophilic slippery coating is taken from the group comprising a patch and a gel.

16. The method for improving intraoral milieu as recited in claim 15, wherein said formulations carried by said hydrophilic slippery coating comprise a sustained release formulation.

17. The method for improving intraoral milieu as recited in claim 15, wherein said hydrophilic slippery coating comprises a patch having a sustained release mouth freshener that is applied to a palatal surface.

18. The method for improving intraoral milieu as recited in claim 15, wherein said hydrophilic slippery coating comprises a gel applied by a device having a geometric configuration complementary to the surfaces to which the gel is applied.

19. The method for improving intraoral milieu as recited in claim 18, wherein said device extrudes a coating into a mucoadherent patch having a geometric configuration complementary to the surfaces to which the gel is applied.

20. The method for improving sustained and localized drug presence by binding to a durable substrate, dendrimers with desirable structural moieties that are therapeutic, and resist elution, metabolic breakdown and excretion.

21. A device having crushable ampoules for delivery of the contents thereof, comprising:
a tubular container;
a crushable ampoule maintained within said tubular container; and
wherein said tubular container has a first inner surface area configured to deliver focused pressure to a surface of said ampoule to effect crushing thereof.

22. The device as recited in claim 21, wherein said first inner surface area configured to deliver focused pressure comprises a pointed ridge.

23. The device as recited in claim 22, wherein said tubular container further has an inner surface configured for structural weakness to yield upon the application of pressure, allowing said pointed ridge to forcefully engage said ampoule.

24. A device for treating aphthous ulcers, comprising:
A motorized handle having a receiving end for removable and replaceable working tips comprising a soft brush, a soft cover, and a soft cleaning tip, wherein at least certain of said tips release medicaments.

25. The device for treating aphthous ulcers as recited in claim 24, wherein at least some of said at least certain of said tips release different medicaments.

26. A mucoadherent material formulation in combination with a medicaiment selected from the group of an antacid, a steroid, and an immune modulator.

27. The mucoadherent material as recited in claim 26, wherein said formulation comprises carboxymthyl cellulose.

28. The mucoadherent material as recited in claim 26, wherein said formulation comprises hydroxyproply cellulose.

29. The mucoadherent material as recited in claim 26, wherein said formulation is cross-linked esterified hydroxyproplyl cellulose.

30. A mucoadherent oral strip, comprising a plurality of layers of dissolvable and digestible materials, said layers carrying active ingredients.

31. The mucoadherent oral strip as recited in claim 30, wherein a first of said layers is faster dissolving than the remainder of said layers.

32. The mucoadherenet oral strip as recited in claim 31, wherein a concentration of said active ingredients is different among the various of said layers.

33. The mucoadherent oral strip as recited in claim 32, wherein at least one layer is formulated to buffer the taste buds and contact mucosa of the mouth from direct stimulation by other of said layers.

34. A method for improving intraoral milieu comprising adhering an intraoral applique, carrying an active ingredient, to a portion of the oral cavity other than the tongue.
